(19) United States
(12) Patent Application Publication
(10) Pub. No.: US 2026/0083393 A1
(43) Pub. Date: Mar. 26, 2026

(54) Method, computer readable medium and computer program for assisting a first user in capturing a digital image of a transparent wound dressing, and for assisting a second user in reviewing digital images of a transparent wound dressing

(71) Applicant: Essity Hygiene and Health Aktiebolag, Goteborg (SE)

(72) Inventors: Sofia Heddson Fransén, Göteborg (SE); Marthin Freij, KALMAR (SE)

(21) Appl. No.: 19/108,814

(22) PCT Filed: Sep. 30, 2022
(86) PCT No.: PCT/EP2022/077390

§ 371 Date: Mar. 5, 2025

(51) Int. Cl. A61B 5/00; A61F 13/00; G16H 30/40

(52) U.S. Cl. CPC A61B 5/445; A61B 5/0077; A61B 5/4842; A61B 5/7425; A61F 13/00; G16H 30/40; A61B 2576/00; A61F 2013/00182

(57) ABSTRACT

The invention relates to a method for assisting a user in capturing a digital image of a transparent wound dressing preferably for monitoring the healing process of a wound dressed with and visible through the transparent wound dressing, to a method for assisting a user in reviewing digital images of a transparent wound dressing, and to corresponding computer programs and computer readable media. Also, a system for assisting a first user in capturing a digital image of a transparent wound dressing and for assisting a second user in reviewing digital images of a transparent wound dressing is described. In order to enhance both user friendliness and reliability of the digital image acquisition, it is proposed to superimpose an overlay image with visual indications on the digital images currently obtained.

### CROSS REFERENCE TO RELATED APPLICATIONS

This application is a National Stage application of PCT/EP2022/077390, filed Sep. 30, 2022, the contents of which are incorporated by reference in their entirety herein.

### TECHNICAL FIELD

The invention relates to a method for assisting a first user in capturing a digital image of a transparent wound dressing, preferably for monitoring the healing process of a wound dressed with and visible through the transparent wound dressing, to a method for assisting a second user in reviewing digital images of a transparent wound dressing and to corresponding computer programs and computer readable media. Also, a system for assisting a first user in capturing a digital image of a transparent wound dressing and for assisting a second user in reviewing digital images of a transparent wound dressing is described.

### BACKGROUND

In the context of the digitalization of services across all disciplines, it is already known to equip health care professionals with mobile hardware and software to support them in their everyday tasks. For example, apps are known that reliably recognize a type of wound based on images of a wound and expert input from the health care person who is on site with the patient. Also, apps are known that are able to suggest a type of plaster or wound dressing that is adapted to a type of wound. Even though such digital tools facilitate the work of health care professionals or nurses, they mostly rely, at least in parts, on dedicated knowledge of these users and are rarely intended for use by patients themselves.

If a patient is released from the hospital soon after the surgery has taken place, it would be convenient if the patient could participate in documenting of the wound healing. However, for obtaining information that can actually be used as a basis for health care, the reproducibility of the acquisition or acquisition conditions is very important. As in general and for most data sets that are aimed to be analysed afterwards, and in particular data sets that should enable a comparison for determining an evolution or change, it is desirable to reduce the number of parameters that are variable and do not belong to the one to be monitored to a minimum. In the concrete case of monitoring an evolution of an item-such as a surgical wound-by analysing digital images showing this item that have been taken successively over time, for instance, it would be ideal that only the pictured item changes from image to image. However, it is precisely because the successive images are taken at different times that it is difficult to reproduce the acquisition conditions from one image to the other. Parameters such as shooting angle, brightness, distance between the used imaging sensor and the item and so on may vary more or less strongly. These difficulties do not only concern patients, but also even trained professionals.

One aid in monitoring wound healing after surgery are wound dressings known from the state of the art, which are at least in part, transparent. Such transparent wound dressings allow observing the state of a wound that they protect without needing to be removed. In this way, the healing process can be followed without altering the beneficial effect of the protection. However, a medical follow-up, even for a wound protected by such a transparent wound dressing, requires a precise collection of information, which is most often carried out by qualified health care staff, e.g., health care professionals at the patient's location. After hospital release, for instance, it is generally seen that qualified personnel takes charge of the collection of information and travels from patient to patient. Alternatively, the patient has to return to a medical facility to allow the medical personnel to monitor the progress of the wound healing on site.

In general, gathering information that is suitable for a diagnostic analysis, in particular gathering sets of information that are easily comparable with each other, is not trivial and in constant need for improvement. Next to the quality of image or data acquisition and the quality of processing or treatment of information, the way the obtained information is provided to, e.g., a health care professional, has a great impact on the quality of the diagnosis. In particular the comparability of the presented data is of utmost importance for the quality of the diagnosis. At present, the data collection for diagnostic purposes is, therefore, mainly done by health care professionals to ensure a high quality of the data. Due to the high workload that this creates for health care professionals, it would be beneficial if the patient could participate in data acquisition without reducing the quality of the subsequent diagnosis.

Accordingly, there is a need for improved systems and methods which are adapted for both patients and health care professionals, and in particular which are adapted for assisting such users in obtaining and/or reviewing information on a wound, in particular in obtaining such information in a reliable way for later analysis.

### SUMMARY

Consequently, it is an object of the present invention to improve existing methods and products. In particular, it is an object of the present invention to provide methods and digital products which enable recording information on a wound in a reliable manner with the aim of providing sufficient information material that is adapted for serving as a basis to a monitoring of the wound over time, and which are user friendly both for trained health care professionals and patients.

Another object of the present invention is to provide a method, a computer program and a computer readable medium that are user friendly for patients with a wound that has been dressed after a medical operation, wherein the healing process of the wound should be monitored further, e.g. after hospital release.

Yet another object of the present invention is to provide a method, a computer program and a computer readable medium that facilitate access of a health care professional to information that has been detected over time and concerning a wound, whose healing process should be monitored.

These objects are achieved by the present invention by a method for assisting a user in capturing a digital image of a transparent wound dressing, preferably for monitoring the healing process of a wound dressed with and visible through the transparent wound dressing, including at least the following steps: obtaining, by means of an imaging device, at least one digital image of the transparent wound dressing, and displaying, by means of a user interface, the obtained at least one digital image of the transparent wound dressing; superimposing an overlay image on the at least one digital image of the transparent wound dressing displayed by the user interface; and saving at least one digital image of the transparent wound dressing.

Superimposing an overlay image on digital images provides the user of the imaging device with indications on how to take a picture. In particular, the overlay image sets a framework for a plurality of acquisition conditions which is intuitive for a user to follow. As an effect, the image acquisition conditions can be easily adapted to capture a digital image with as little deviation as possible from the indications given by the overlay image.

This way, a patient is enabled to obtain by himself an image of a transparent wound dressing he is wearing and meet, at least in parts, predetermined specifications for the image acquisition conditions. As the wound dressing is transparent, the acquired image also shows the wound underneath, so that the information of digital images obtained in this way are suitable as a basis for a medical analysis afterwards.

A digital image may be a data set including information on an image composed of picture elements, also known as pixels, each with finite, discrete quantities of numeric representation for its intensity, a gray level, and/or a color code, wherein each picture element or pixel has an allocated spatial position within an image surface defined by a two-dimensional function with spatial coordinates denoted with x, y on the x-axis and y-axis, respectively. A digital image may be captured by acquiring information on light intensity, gray levels, and/or a color described with a color code, e.g. as a coordinate in the RGB color space, as a function of spatial position over a predetermined surface via a light sensor and storing, at least temporary, the acquired information on a computer readable memory. In this case, the picture element positions or pixel positions within the digital image correspond to spatial positions within the light sensible surface of the light sensor.

Obtaining the at least one digital image is performed by means of an imaging device. Such an imaging device may include at least one sensor, at least one processor, and at least one memory, wherein the at least one sensor, the at least one processor, and the at least one memory are connected with each other for transferring information. The at least one sensor may have a predetermined acquisition surface, which is sensible to light or at least to a range of electromagnetic waves, be configured for acquiring light intensity, a gray level, and/or a color code as a function of spatial position over its acquisition surface and be configured for transferring acquired information to the memory. The processor may be configured for reading information from the memory and transforming read information into corresponding digital signals.

The user interface may have at least one display and be connected to the imaging device. The display may be configured for processing digital signals corresponding to digital images into visible-light images and present the visible-light images. Further, the user interface may have at least one user input detector and a processor that are configured for obtaining a user input and execute a command corresponding to the user input. This way, a user to whom the at least one obtained digital image is displayed may interact with the user interface and enter, as a reaction, a user input with instructions. As a matter of example, a user input may correspond to an instruction to trigger saving the displayed digital image.

The user interface and the imaging device may be combined into one single device. As a matter of example, a smartphone, a tablet, a laptop or any other mobile device having a camera and a display may be used in the present method for both obtaining the at least one digital image and displaying the obtained digital image. Preferably, the mobile device used in the method of the invention is a smartphone or a tablet.

The at least one digital image is an image of a transparent wound dressing. In other words, the acquired information containing light intensity, a gray level, and/or a color code as a function of a spatial position corresponds, at least in parts, to a representation of a transparent wound dressing.

In the context of the present invention, a transparent wound dressing may be any kind of item enabling a protection of wound, and which is, at least in parts, transparent. A “wound” in the present context may be any type of open wound, such as a laceration of the skin. For example, the wound can be a surgical wound, i.e. a wound caused by a surgical intervention. The wound can also be a laceration caused by accident. The wound dressing is a medical dressing that is preferably able to absorb fluids, such as wound fluids. It is, thus, preferably an absorbent dressing. The wound dressing can be adapted to be used on skin, e.g. human skin, and will, consequently adhere to the skin of the wearer, e.g. to the skin of a patient. To be able to absorb wound fluids, the wound dressing can include one or more absorbent bodies. The wound dressing can additionally include further layers with various properties-such as layers which promote adherence to the skin or enhance the integrity of the dressing and/or layers which have additional functions, such as layers which are suitable to enhance the wound healing process by providing additional absorption capacity or releasing active agents which support the wound healing process.

The wound dressing can, for example include or consist of (a) a covering layer; (b) the absorbent body or bodies on the wound-facing side of the covering layer, and (c) a wound-contact layer on the wound facing side of the one or more absorbent bodies. The wound dressing can, moreover, include an adhesive layer on the wound facing side of the wound-contact layer. It will be understood that all components of the wound dressing can be transparent in toto or in parts to facilitate the required transparency of the wound dressing.

The term “transparent” or “transparency” refers to the property of transmitting light, at least with some degree of transmission of light from one side to another. A degree of transmission can vary from total transmission to opacity. However, the digital images that are collected in the context of the present invention preferably show a wound underneath, so as to allow a monitoring of the healing process of the wound. Therefore a degree of light transmission is preferably chosen as to allow for seeing through the wound dressing. Specifically, a “transparent” wound dressing in the present context indicates that features (e.g. a wound) on a surface immediately beneath the wound dressing (e.g. the skin of a patient) would be visible through the wound dressing (e.g. when viewed by a human at a distance of 25 cm). The wound dressing may be completely transparent, i.e. over its entire surface, or may have one or more transparent sections which enable the user to view the underlying wound.

The overlay image may be a digital data set corresponding to information on light intensity, a gray level, and/or a color code as a function of spatial position over a predetermined surface. Accordingly, the overlay image may be a coloured image, and/or a black and white image. Also, the overlay image is configured for being, at least visually, superimposed over at least one digital image. In the framework of the present invention, the term superimposing shall encompass displaying a digital image and an overlay image together at the same time, wherein the spatial positions of the image elements or pixels of both the digital image and the overlay image are matched. Preferably, the overlay image is configured with a level of transparency that allows a digital image, over which the overlay image is superimposable, to be seen through.

In one embodiment, at least two digital images of the transparent wound dressing are obtained by means of the imaging device, the at least two digital images of the transparent wound dressing are displayed, by means of the user interface, as a live feed, and the overlay image is superimposed on the live feed displayed by the user interface.

With this, the overlay image gives an indication of what the digital image to be saved should look like, as well as giving the possibility to change the current acquisition conditions. As a result, it helps a user to adapt in real time and in situ, for example, the position or angle of shooting of the imaging device to obtain a digital image in the desired condition. In particular, it allows a user to be guided in real time to predetermined acquisition conditions in an intuitive way.

A live feed in the sense of the invention may be a representation of a sequence of digital images that have been acquired successively within the immediate past or at least in the course of very few time units before representation. In particular, the term “live feed” encompasses the form of a real-time video.

According to another embodiment, the overlay image includes at least one visual indication on the appearance the transparent wound dressing should, at least in part, exhibit on the at least one digital image of the transparent wound dressing to be saved.

Superimposing an overlay image with, e.g., an indication on the size the transparent wound dressing is expected to show on a digital image to be saved, serves as reference for adapting the distance between the imaging device and the transparent wound dressing, so as to match the desired size pattern as far as possible.

The at least one visual indication may be a representation of at least one piece of information about a size, a length, a width, a thickness, a distance, a size ratio, a shape, a curve, an outline, an angle, a colour, an aspect, a pattern, a contrast, a position, a type of transparent wound dressing, or a combination thereof. Specifically, the visual indication can be a shape of a wound dressing including the shape of structural and/or sub-structural elements of the wound dressing, such as, e.g. gel pads or edge zones with adhesion function. The visual indication may be represented as a schematic representation, a drawing, a photographic picture. Such a schematic representation may consist of or include a frame, a partial frame, a line among others. The schematic representation may be adapted to the contours of the wound dressing used, e.g., have the same shape as the wound dressing when worn and viewed from a distal direction in the direction of the skin. The schematic representation, e.g. in the form of a frame or partial frame, can have an oval shape, a round shape, an elliptic shape, a rectangual shape, a cloverleaf-like shape or one or more parts of these shapes. One or more parts of these shapes, could e.g. be two parts of these shapes, e.g. of an oval shape, showing e.g. only 5-30% of the distal part of the shape and 5-30% of the proximal part of the shape.

According to a further embodiment, the at least one visual indication has an aspect ratio that is the same or substantially the same as an aspect ratio of the transparent wound dressing of which a digital image should be saved.

This allows assisting a user in adjusting the position and angle of the imaging device with respect to the transparent wound dressing, so that the imaging device is then positioned to acquire a digital image as desired. In particular, displaying an overlay image with a visual indication having a particular aspect ratio restricts the range of possible positions of the imaging device for a correct acquisition. Correspondingly, the overlay image serves as precise and simple indication for guiding a user towards expected acquisition conditions.

An aspect ratio of the shape of the transparent wound dressing can be understood as a proportion or size relation between outlines of the shape of the transparent dressing. Specifically, the aspect ratio of a rectangle shape is the ratio of its sizes in different dimensions. For example, the aspect ratio of a rectangle is the ratio of its longer side to its shorter side—the ratio of width to height, when the rectangle is oriented as a “landscape”. For an ellipse, the aspect ratio denotes the ratio of the major axis to the minor axis.

In exemplary embodiments, the aspect ratio of the wound dressing's shape represented on the overlay image and the shape itself may be the only information needed to produce a schematic representation for the overlay image.

According to another embodiment, the at least one digital image of the transparent wound dressing is saved in association with a chronological indicator.

As a beneficial effect, the time of acquisition of the digital image can be taken into account for a later analysis. When several digital images are saved, they can then be placed in the order of their respective time of acquisition. Overall, this feature enables monitoring the evolution of the transparent wound dressing and of a wound dressed with the transparent wound dressing.

The chronological indicator may be a place of order of a digital image in a sequence of digital images, a time stamp indicating the time at which the at least one digital image was saved, a number created by iterative process in counting the saved digital images, a chronological order in a serialized list.

According to another embodiment, the method further includes, before superimposing the overlay image on the at least one digital image of the transparent wound dressing displayed by the user interface, checking if at least one digital image of the transparent wound dressing has been saved in association with a past chronological indicator; if yes, using one of the at least one digital images of the transparent wound dressing saved in association with a past chronological indicator for obtaining the overlay image; and if not, retrieving and using information on the appearance the transparent wound dressing should, at least in parts, exhibit on the at least one digital image to be saved for obtaining the overlay image.

Accordingly, several digital images of a wound dressed with a transparent wound dressing can be obtained reliably over time with similar acquisition conditions, regardless of the level of expertise of the user assisted by the method. In particular, a first digital image can be obtained with at least approximatively acquisition conditions as indicated by the visual indication from the overlay image displaying a first overlay image as a drawing or schematic representation having visual indication about the shape of the transparent wound dressing, and then further digital images of the transparent wound dressing can be obtained with detection conditions that are similar to the ones of the first image by guiding the user with the first digital image itself as transparent overlay image.

Checking if digital images have been saved in the past can be performed by retrieving information about digital images saved on a memory and analysing the corresponding chronological indictors, if any.

Retrieving information on the appearance the transparent wound dressing should exhibit on the image to be saved can be performed by receiving information from a memory storing information issued from earlier user inputs or obtained via a method for automatic or semi-automatic recognition of a wound dressing. In particular, the information on the appearance the transparent wound dressing may include the output of a computer program executed upstream of the present method for recognizing or suggesting a type of wound dressing. Alternatively or in complement, retrieving information on the appearance of the transparent wound dressing may be performed by presenting a corresponding question to the user via the user interface, collecting a user input, processing the user input and creating an overlay image with a visual indication corresponding to the user input or retrieving a prepared overlay image from a databank with a visual indication corresponding to the user input. For example, the user could receive a question on the type of wound dressing that is used, wherein the “type” can be defined e.g. by the manufacturer, the tradename, the size, the shape and/or other information that is suitable to characterize the wound dressing, in particular the shape and size of the wound dressing.

Using a digital image of the transparent wound dressing saved in association with a past chronological indicator for obtaining the overlay image may include saving a copy of the past digital image, modifying its characteristics so that the user interface displays it with a higher level of transparency as a digital image on which the overlay image should be superimposed on, and saving the modified copy as overlay image. While using a combination of a least two of the at least one digital image of the transparent wound dressing saved in association with a past chronological indicator, the characteristics of these digital images may be gathered, and processed for creating an overlay image with characteristics corresponding to a progression of the characteristics of the past digital images and having a higher level of transparency as a digital image the overlay image should be superimposed on.

According to another embodiment, saving at least one digital image of the transparent wound dressing is automatically triggered as a consequence of the result of a comparison between characteristics of at least one of the at least one digital images obtained by the imaging device and characteristics of the overlay image.

Such an automatic triggering increases both the user friendliness and the reliability of the method. A digital image acquired in desired acquisition condition may be saved automatically. Thereby the number of interventions a user performs himself is reduced. Moreover, the image acquisition is triggered on the basis of predetermined objective criteria, and, as such, not dependent on the judgement of a user.

The characteristics that are compared may, e.g., be chosen from the group consisting of: pixel position of a contrast, exceeding or undercutting of a threshold for light intensity or grey level at a particular pixel position, distance between contrasts. The term contrast is meant here as difference in luminance or colour that makes an object (or its representation in an image or display) distinguishable. In terms of visual perception, a contrast may be determined by the difference in the colour and brightness of the object and other objects within the same field of view. Therefore, comparing the pixel position of a particular high contrast in the digital image with the pixel position of a similarly high contrast in the overlay image, a match of object position, e.g. of the transparent wound dressing, can be recognized.

Saving the at least one digital image may be triggered based on the recognition of a match between characteristics of a part of the digital image and characteristics of the overlay image. For instance, a digital image, for which the transparent wound dressing is represented with a smaller size as in the overlay image, but a correct position, lightning etc., may be recognized as matching with the overlay image. This may be executed by considering zoomed versions of the digital images for the comparison with the overlay image.

It is possible to make use of an algorithm for object recognition in a digital image, or e.g., of an artificial intelligence model than can be trained in recognizing a transparent wound dressing or a wound.

According to another embodiment, saving at least one digital image of the transparent wound dressing is automatically triggered after elapse of a predetermined time duration.

Such an automatic triggering also elevates the user friendliness, and enables a simple design of the method and well as of the devices implicated in the method.

For instance, the predetermined time duration may begin with the start of displaying the overlay image, with the begin of obtaining at least one digital image via the imaging device, with detecting a user input with a corresponding command, or with automatically recognizing a match between characteristics of the overlay image and characteristics of at least one obtained digital image.

According to another embodiment, the method further includes outputting a first signal when the result of a comparison between characteristics of at least one of the digital images obtained by the imaging device and characteristics of the overlay image falls within a predetermined range for a degree of similarity; and/or outputting a second signal when the result of the comparison between characteristics of at least one of the at least one digital image obtained by the imaging device and characteristics of the overlay image falls outside the predetermined range for a degree of similarity. The first signal can be a pointer for the user that conditions are not optimal for image acquisition while the second signal can be a pointer for the user to trigger the image acquisition.

With the signals, the user can be guided on the basis of objective criteria used for the comparison. Accordingly, the user assistance as well as the probability of success are enhanced.

The predetermined range for a degree of similarity can be stored on a memory and be based either on earlier user input or information given by a producer or programmer of devices implicated in the method. The predetermined range may include information on at least one parameter value corresponding to at least one of the characteristics the comparison is based on, as well as at least a tolerance range for the respective at least one parameter value. A degree of similarity may correspond to a difference obtained by comparing corresponding characteristics of a digital image and the overlay image. Correspondingly, a high degree of similarity would be a relatively low value difference for the compared characteristics, while a low degree of similarity would be a relatively high value difference for the compared characteristics. The range for a degree of similarity allows for an objective judgement of whether the degree of similarity is high or low.

The first signal is intended to indicate the situation where the position or acquisition conditions in general of the imaging device are too far from the expected acquisition conditions set by the overlay image, while the second signal is intended to indicate the case when a match between the overlay image and at least one obtained digital image is recognized.

According to another embodiment, the method further includes automatically adapting the settings of the imaging device based on the result of a comparison between characteristics of at least one of the at least one digital image obtained by the imaging device and characteristics of the overlay image.

Also this feature enhances the user friendliness and the chances of success further at the same time, since a user intervention is avoided by the automatic setting of the imaging device. Moreover, the adaptation of the acquisition condition via adjustment of the device settings provides an additional approach for meeting expected acquisition condition.

The settings of the imaging device may e.g. be selected from the group consisting of a focal length, a focusing distance, an exposure time, an aperture of a lens, a sensitivity of the sensor to light or ISO, a stability or a movement of the imaging device, an optical stability of a lens or sensor, an image file format, a white balance, a contrast of light tones compared to dark tones, a color saturation or color space, a sharpness, an image compression which impacts the size of the image file, and a brightness.

According to another embodiment, the method further includes obtaining identification information allocated to a patient wearing the transparent wound dressing and/or to the transparent wound dressing; and saving the at least one digital image of the transparent wound dressing in association with the obtained identification information.

This allows for allocating the at least one saved digital image and, accordingly, the state of the wound that may be visible through the transparent wound dressing represented on the digital image, to a particular patient or patient file. In particular, several digital images saved with the same identification information or with parented identification information relating to a same wound or patient can be gathered for a later analysis and monitoring of a healing process.

The term “identification information” is to be understood as including person-related data such as a name, surname, date of birth, gender, address, dates of a hospital journey, information on a medical intervention, medication, and similar, as well as a code, such as a code number or letter sequence, that would allow for anonymization. Such information may be a computer-generated unique code that can be translated into person related data when processed together with encoding data.

According to another embodiment, the method further includes obtaining wound information on a wound dressed with the transparent wound dressing; and saving the at least one digital image of the transparent wound dressing in association with at least one piece of the obtained wound information.

This embodiment allows for gathering complementary information on the wound dressed with the wound dressing, so as to enable a better monitoring of the healing process of the wound later on by a qualified person receiving the output of the present method.

It is to be noted that the method described here is not a therapeutic or diagnostic method, but rather a method for gathering and processing information that may be used by health care persons in a separate diagnostic method later-on. This applies in an equal manner to the computer readable media, the computer programs described therein. It is also possible to perform the method with an additional diagnostic step by reviewing the at least one saved digital image of the transparent wound dressing. In particular, the review may be performed by comparing the at least one saved digital image with at least one previous digital image of the transparent wound dressing.

Wound information may, e.g., be chosen from the group consisting of: color of the wound, body temperature, temperature in the wound area, appearance of the wound area, whether fluid leaks from the wound, whether the wound is closed or opened, whether the transparent wound dressing has been replaced.

Wound dressing information may be obtained as user input, e.g. by means of the user interface, or via an upstream computer program or method for determining wound information.

The above-mentioned objects are also achieved according to the invention by a computer program including instructions, which, when executed by an apparatus, causes the apparatus to perform the method described above. Preferably, the apparatus is a mobile device, in particular a smartphone.

The above-mentioned objects are also achieved according to the invention by a computer readable medium including program instructions stored thereon for performing the method according to the invention. Preferably, the apparatus is a mobile device, in particular a smartphone.

The above-mentioned objects are also achieved according to the invention by a method for assisting a user in reviewing digital images of a transparent wound dressing, including at least the following steps: obtaining at least two digital images of the transparent wound dressing captured according to the method described above, wherein each digital image of the transparent wound dressing is associated with a respective chronological indicator; providing a slider, wherein the slider can adopt at least as many positions as digital images of the transparent wound dressing have been obtained, and wherein one position of the slider is assigned to one chronological indicator of the obtained at least two digital images of the transparent wound dressing; and displaying the one digital image of the at least two digital images of the transparent wound dressing whose chronological indicator is assigned the current position of the slider.

This method preferably makes use of digital images obtained with the method for assisting a user in capturing a digital image of a transparent wound dressing as described above and is complementary hereto. With the present method, digital images can be placed into order according to the order or time of their respective acquisition. This way, the digital images can actually be used for an analysis of an evolution of the wound visible through the transparent wound dressing represented on the digital images. Accordingly, the user reviewing the digital images may be a health care professional (HCP), e.g., a HCP in a location remote from the patient's location, such as in a hospital. The method enables the HCP to remotely monitor the progress of the wound healing.

The slider may be a movable cursor, wherein the cursor can adopt at least two different positions within a predetermined position range. In particular, the movable cursor may be movable along one axial direction in accordance with a user input and the position range include at least two positions that differ in a coordinate value on the axial direction.

Specifically, a transition between digital images with successive chronological indicators may be displayed in such a way that the digital images blend in, while the cursor is moved from a first position to a subsequent position.

Alternatively, it is possible to display only the digital image with the chronological indicator assigned to the current position of the slider's cursor.

The above-mentioned objects are also achieved according to the invention by a computer program including instructions, which, when executed by an apparatus, causes the apparatus to perform the method for assisting a user in reviewing digital images of a transparent wound dressing as described above.

The above-mentioned objects are also achieved according to the invention by a computer readable medium including program instructions stored thereon for performing the method for assisting a user in reviewing digital images of a transparent wound dressing as described above.

The above mentioned objects are also achieved according to the invention by a system for assisting a first user in capturing a digital image of a transparent wound dressing and for assisting a second user in reviewing digital images of a transparent wound dressing, including at least the following: a first mobile device having a first computer readable medium as described above, an imaging device and a user interface; a second device having a second computer readable medium as described above and a second user interface; and a server adapted for obtaining from the first mobile device at least one, preferably at least two, digital images of a transparent wound dressing captured by the first mobile device, for storing the obtained at least one digital image of a transparent wound dressing captured by the first mobile device, and for transmitting the stored at least one digital image of a transparent wound dressing captured by the first mobile device to the second device.

Such a system is configured for, on the one side, assisting a user in capturing a digital image of a transparent wound dressing, storing and transferring the captured digital images, and on the other side, assisting another user in reviewing the digital images of a transparent wound dressing. Overall, the system allows for obtaining in a reliable manner digital images that have been obtained in acquisition conditions that are sufficiently similar for a comparison of the digital images, and also for ordering and the digital images in comprehensible way. With this, a patient can furnish a health care professional (HCP) with actually usable information and the health care professional is put into a situation adequate for monitoring the healing process of a wound.

The first mobile device and the second device may be one single device having all features of both the mobile device and the second device as described above. This way, both obtaining digital images of the transparent wound dressing in a reliable way and providing the obtained digital images in a user friendly way are possible with one device. As an example, a health care professional is then able to visit a patient, save digital images of the transparent wound dressing at different dates, and subsequently review the saved digital images for establishing a diagnosis.

### BRIEF DESCRIPTION OF THE FIGURES

FIG. 1 shows a flowchart of a method for assisting a user in capturing a digital image of a transparent wound dressing;

FIG. 2 shows a computer readable medium performing a first step of the method for assisting a user in capturing a digital image of a transparent wound dressing;

FIG. 3 shows the computer readable medium of FIG. 2 performing a further step of the method for assisting a user in capturing a digital image of a transparent wound dressing;

FIG. 4 shows the computer readable medium of FIG. 2 performing a further step of the method for assisting a user in capturing a digital image of a transparent wound dressing;

FIG. 5 shows a flowchart of a method for assisting a user in capturing a digital image of a transparent wound dressing according to a second embodiment;

FIG. 6 shows a flowchart of a method for assisting a user in capturing a digital image of a transparent wound dressing according to a third embodiment;

FIG. 7 shows a flowchart of a method for assisting a user in capturing a digital image of a transparent wound dressing according to a fourth embodiment;

FIG. 8 shows a further computer readable medium performing a step of a method for assisting a user in capturing a digital image of a transparent wound dressing according to the fourth embodiment;

FIG. 9 shows flowchart of a method for assisting a user in capturing a digital image of a transparent wound dressing according to a fifth embodiment;

FIG. 10 shows flowchart of a method for assisting a user in capturing a digital image of a transparent wound dressing according to a sixth embodiment;

FIG. 11 shows flowchart of a method for assisting a user in capturing a digital image of a transparent wound dressing according to a seventh embodiment;

FIG. 12 shows a computer readable medium performing a step of a method for assisting a user in reviewing digital images of a transparent wound dressing; and

FIG. 13 shows a system for assisting a first user in capturing a digital image of a transparent wound dressing and for assisting a second user in reviewing digital images of a transparent wound dressing.

### DETAILED DESCRIPTION

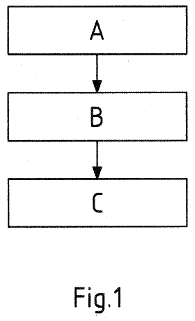

With reference to FIG. 1, three steps A, B and C of a method for assisting a user in capturing a digital image of a transparent wound dressing are represented. In step A, at least one digital image of a transparent wound dressing are obtained by means of an imaging device and displayed by means of a user interface. At step B, an image representing schematically the specific type of the transparent wound dressing is superimposed as an overlay image on the digital image via the display. In other words, the digital image obtained by means of the imaging device and the overlay image are displayed together via the user interface. Subsequently, the digital image is saved at step C.

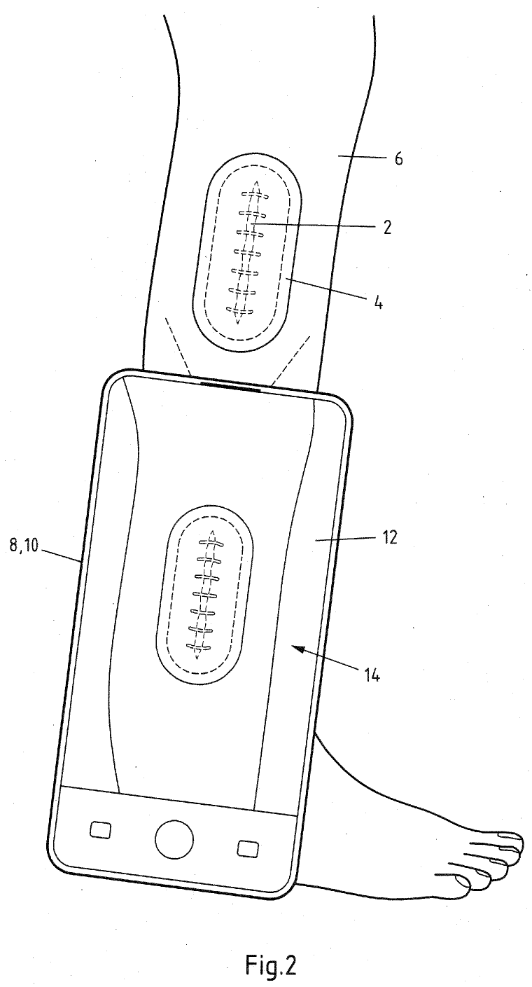

Turning to FIG. 2, a wound 2, a transparent wound dressing 4 worn by the wounded patient 6 and a smartphone 8 with an imaging device (not shown due to the perspective of the represented view) and a user interface 10 including a display 12 are shown. The smartphone 8 is performing step A of the method of FIG. 1. Accordingly, the imaging device acquires a digital image 14 of the wound 2 and of the transparent wound dressing 4 and the user interface 10 displays the acquired digital image 14.

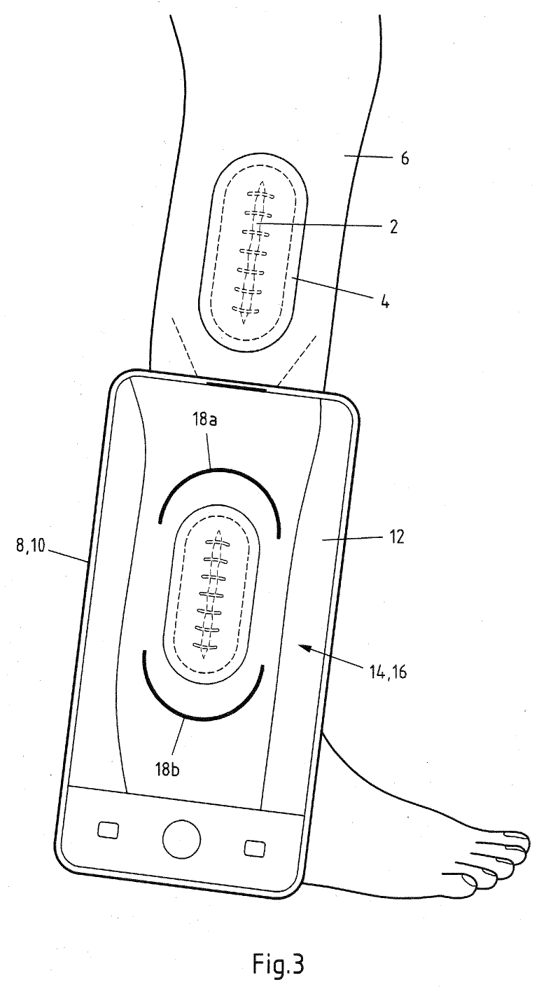

In FIG. 3, the user interface 10 of the smartphone 8 displays the acquired digital image 14 and an overlay image 16 superimposed on the digital image 14, according to method step B of the method represented in FIG. 1. The overlay image 16 is transparent with the exception of a schematic representation of curved distal and proximal ends 18a, 18b of a transparent wound dressing 4, thereby indicating the desired size and position the transparent wound dressing 4 should exhibit on a digital image 14 to be saved. In the present case, the overlay image 16 has been created by retrieving a basic image with a basic schematic representation of curved distal and proximal ends 18a, 18b of a transparent wound dressing 4 from a data bank, retrieving the information on the size of the wound dressing 4, processing both the retrieved basic image and the size information for producing the overlay image 16 with the schematic representation of curved distal and proximal ends 18a, 18b of a transparent wound dressing 4, wherein the aspect ratio of the schematic representation is adapted to the retrieved size information.

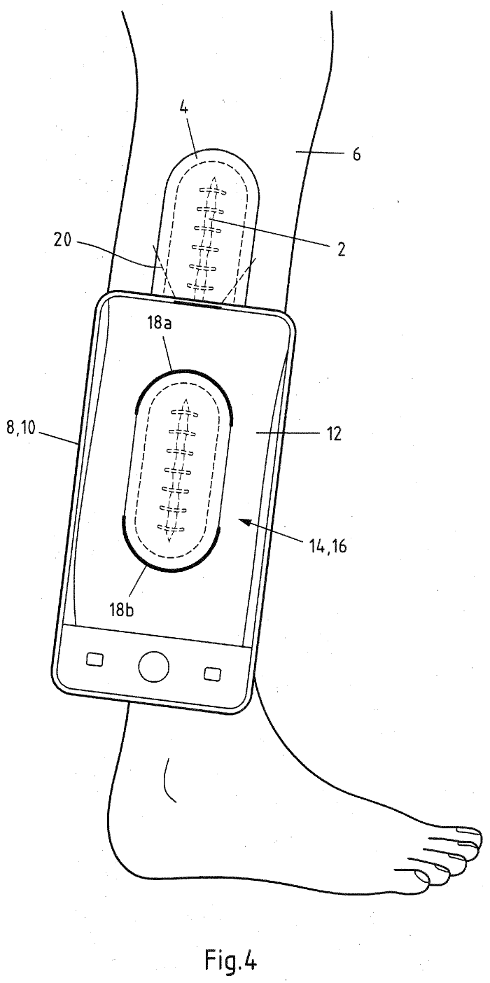

FIG. 4 shows the smartphone 8 of the precedent figure, wherein the acquisition condition for acquiring a digital image 14 by means of the imaging device are adjusted to the indication given by the overlay image 16. More specifically, the distance 20 between the imaging device of the smartphone 8 and the transparent wound dressing 4 has been reduced so as to let the transparent wound dressing 4 appear larger on the display 12.

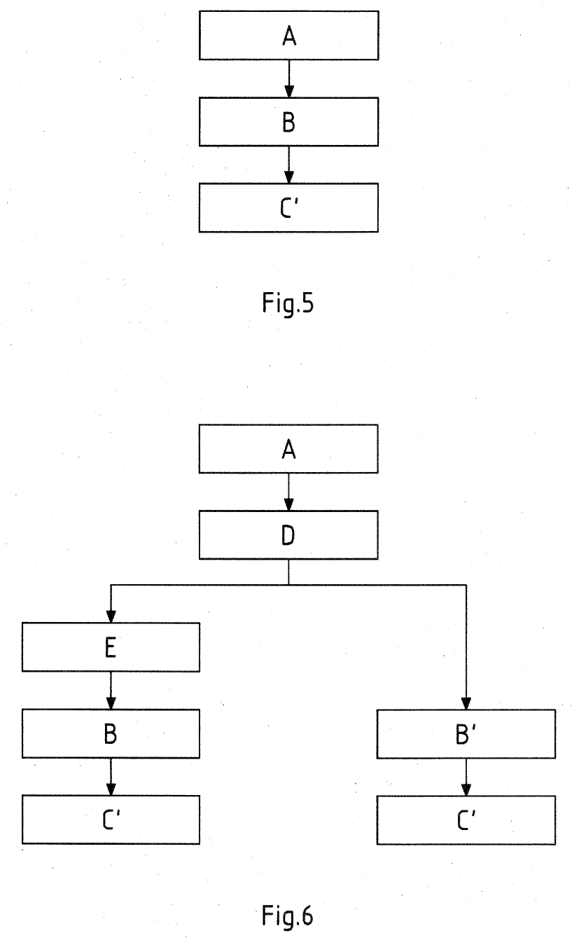

Now turning to FIG. 5, which shows a second embodiment of the method for assisting a user in capturing a digital image of a transparent wound dressing, three steps marked A, B and C′ are represented. Step A and B are the same as steps A and B in FIG. 1. At step C′, the digital image obtained by means of the imaging device in step A is saved together with a chronological indicator. Here the chronological indicator is a time stamp outputted by a clock that records the time at which the digital image has been acquired by the imaging device.

The flowchart of FIG. 6 exhibits several steps of a method for assisting a user in capturing a digital image of a transparent wound dressing according to a third embodiment. The method begins with the same step A as the method in FIG. 1. Then, in step D, a checking if at least one digital image of the transparent wound dressing has been saved in association with a past chronological indicator is performed. Depending on the result, step E or B′ is started.

If at least one digital image of the transparent wound dressing has been saved in association with a past chronological indicator, at least one theses digital images or a combination thereof is used for creating an overlay image in step B′. Here the overlay image is created by duplicating a digital image with the newest chronological indicator, adjusting its settings for being displayed as semi-transparent via the user interface and saving the modified duplicate as overlay image.

If no digital image of the transparent wound dressing has been saved in association with a past chronological indicator, information on the appearance that the transparent wound dressing should, at least in parts, exhibit on the at least one digital image to be saved are retrieved in step E. To do this, a question can be displayed to the user via the user interface and a user input can be obtained, containing information on the type of the transparent wound dressing. Then a prepared overlay image containing a schematic representation of the transparent wound dressing as displayed in step B of FIG. 1 is retrieved from a data bank accordingly to the information contained in the obtained user input. Alternatively, the type of the transparent wound dressing can be recognized in an automated manner. In subsequent step B, the overlay image is superimposed on the digital image obtained in step A. In step C′, the digital image is saved together with a corresponding chronological indicator.

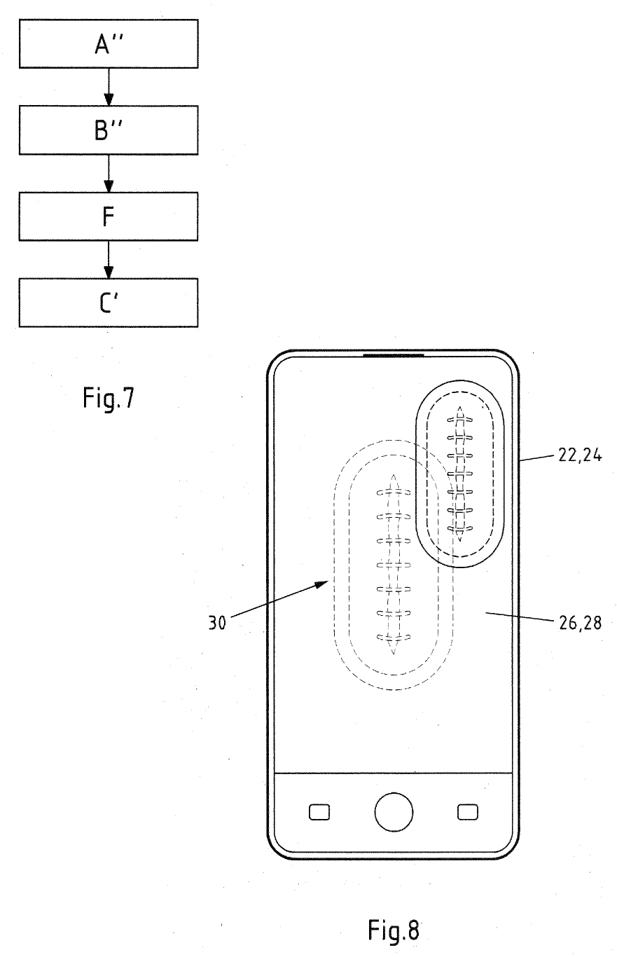

FIG. 7 shows a flowchart of a method for assisting a user in capturing a digital image of a transparent wound dressing according to a fourth embodiment, wherein after obtaining of at least two digital images of the transparent wound dressing by means of an imaging device, these are displayed as a live feed by means of a user interface (step A″). Subsequently in step B″, an overlay image created on the basis of a previously obtained digital image of the transparent wound dressing and saved with a past chronological indicator is superimposed on the live feed. In step F, the digital images of the live feed are compared, respectively, with the overlay image. More specifically, the size and position of the transparent wound dressing of each digital image of the live feed is compared with the size and position of the transparent wound dressing of the overlay image. In step C′, one digital image, for which the size and position of the transparent wound dressing correspond to the size and position of the transparent wound dressing of the overlay image is saved together with a chronological indicator.

FIG. 8 shows a smartphone 22 with a user interface 24 and an imaging device (not shown due to the perspective of the represented view). The user interface 24 includes a display 26 that displays a live feed 28 of digital images 2 of a wound dressing obtained by means of the imaging device and a superimposed overlay image 30. The overlay image 30 is a duplicate of a previously saved digital image with a past chronological indicator, which characteristics have been adjusted for being displayed as semi-transparent.

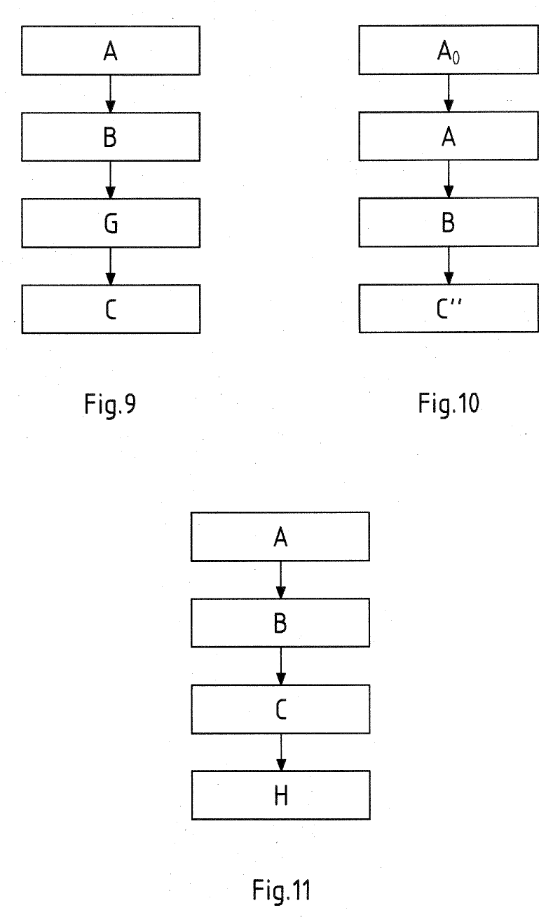

Referring to FIG. 9, which shows a flowchart of a method for assisting a user in capturing a digital image of a transparent wound dressing according to a fifth embodiment, steps A, B and C are the same as steps A, B and C of FIG. 1. After step B and before step C, step G is performed, wherein a predetermined time duration is started, and at the end of which time duration step C of saving the digital image is triggered.

The flowchart of FIG. 10 showing a sixth embodiment of the method exhibits a method step A₀ performed previous to step A. In step A₀, identification information allocated to a patient wearing the transparent wound dressing and/or to the transparent wound dressing are obtained, e.g., as user input while registering for the first time in a corresponding digital application and saved in a memory. At step C″ following step B of superimposing an overlay image on at least one digital image, the digital image is saved together with both a chronological indicator and at least one piece of the identification information obtained in step A₀.

The seventh embodiment of the method as shown in FIG. 11 includes the method steps A, B and C as described for FIG. 1 and a subsequent step H. Step H corresponds to obtaining wound information, here as a user input including information on the appearance of the wound dressed with the wound dressing and the temperature of the wound dressed with the wound dressing, and saving it together with the digital image.

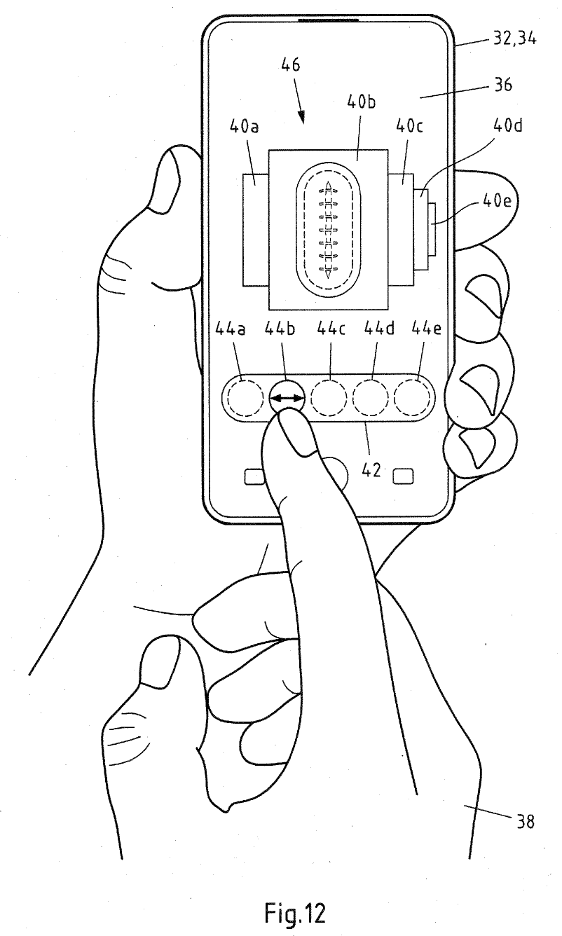

In FIG. 12, a computer readable medium 32 under the form of a smartphone 34 with a display 36 is shown while performing a step of a method for assisting a user 38 in reviewing digital images 40a, 40b, 40c, 40d, 40e of a transparent wound dressing. The display 36 displays a slider 42 adapted for adopting several slider positions 44a, 44b, 44c, 44d, 44e when moved from right to left or from left to right, and digital images 40a, 40b, 40c, 40d, 40e of the transparent wound dressing. The slider 42 is a continuous slider. This means that the slider 42 moves continuously from left to right when the user's thumb is on the slider 42. When the user lets go of the slider 42, the slider 42 snaps to the next possible discrete position that is assigned to specific picture, e.g. from a position intermediate between the first position 44a and the position 44b, it will snap to the next one 44b. In the displayed embodiment, the digital images 40a, 40b, 40c, 40d, 40e are displayed as a carousel 46 (showing the present picture in the centre and adjacent images next to it on the left and right side), wherein the digital image 40b in the front position of the carousel 48 has a chronological indicator corresponding to the current slider position 44b. It will be understood that the digital images do not need to be displayed in the form of such a carousel. Alternatively, e.g., only the image that is presently viewed is shown.

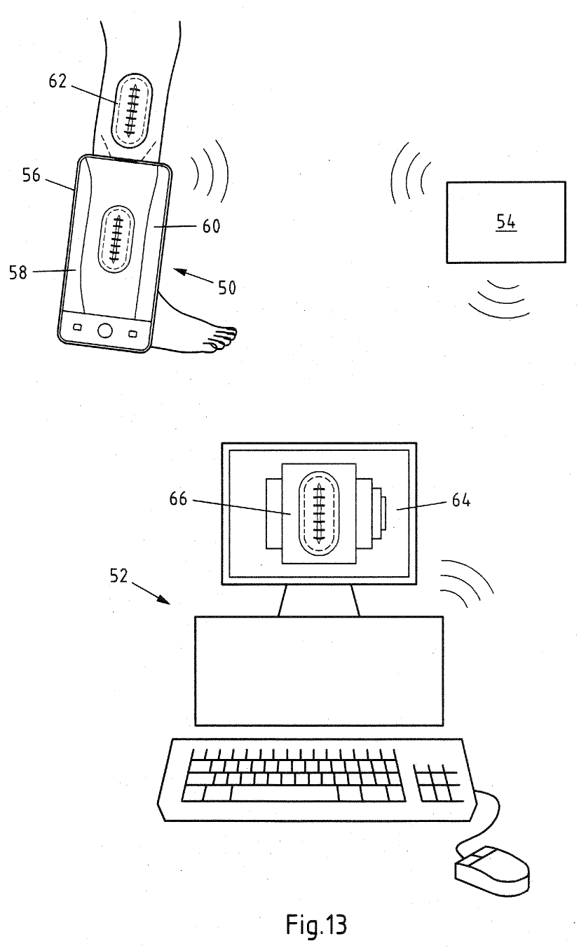

Turning to FIG. 13, a system including a first mobile device 50, a second device 52 and a server 54 is represented. The first mobile, the server 54 and the second device 52 are configured for exchanging information via wireless technology. The first mobile has an imaging device 56 and a display 58 and is configured for performing a method for assisting a user in capturing a digital image 60 of a transparent wound dressing 62 according to at least one embodiment as described previously. The second device 52 has a display 64 and is configured for performing a method for assisting a user in reviewing digital images 66 of a transparent wound dressing 62 according to at least one of the embodiments described previously. In this example, the server 54 is hosted remotely from the first mobile device 50 and from the second device 52 and configured for obtaining from the first mobile device 50 at least one digital image 60 of a transparent wound dressing 62 captured by the first mobile device 50, for storing the obtained at least one digital image 60 of a transparent wound dressing 62 captured by the first mobile device 50, and for transmitting the stored at least one digital image 60 of a transparent wound dressing 62 captured by the first mobile device 50 to the second device 52.

1) A method for assisting a user in capturing a digital image of a transparent wound dressing, preferably for monitoring the healing process of a wound dressed with and visible through the transparent wound dressing, comprising at least the following steps:
obtaining, by means of an imaging device at least one digital image of the transparent wound dressing, and displaying, by means of a user interface the obtained at least one digital image of the transparent wound dressing;
superimposing an overlay image on the at least one digital image of the transparent wound dressing displayed by the user interface; and
saving at least one digital image of the transparent wound dressing.

2. The method according to claim 1,
wherein at least two digital images of the transparent wound dressing are obtained by means of the imaging device,
wherein the at least two digital images of the transparent wound dressing are displayed, by means of the user interface, as a live feed, and
wherein the overlay image is superimposed on the live feed displayed by the user interface.

3. The method according to claim 1, wherein the overlay image comprises at least one visual indication on the appearance the transparent wound dressing should, at least in part, exhibit on the at least one digital image of the transparent wound dressing to be saved.

4. The method according to claim 3, wherein the at least one visual indication has an aspect ratio that is the same or substantially the same as an aspect ratio of the transparent wound dressing of which a digital image should be saved.

5. The method according to claim 1, wherein the at least one digital image of the transparent wound dressing is saved in association with a chronological indicator.

6. The method according to claim 1, further comprising,
before superimposing the overlay image on the at least one digital image of the transparent wound dressing displayed by the user interface, checking if at least one digital image of the transparent wound dressing has been saved in association with a past chronological indicator;
if yes, using one of the at least one digital images of the transparent wound dressing saved in association with a past chronological indicator; and
if not, retrieving and using information on the appearance the transparent wound dressing should, at least in parts, exhibit on the at least one digital image to be saved for obtaining the overlay image.

7. The method according to claim 1, wherein saving at least one digital image of the transparent wound dressing is automatically triggered as a consequence of the result of a comparison between characteristics of at least one of the at least one digital image obtained by the imaging device and characteristics of the overlay image.

8. The method according to claim 1, wherein saving at least one digital image of the transparent wound dressing is automatically triggered after elapse of a predetermined time duration.

9. The method according to claim 1, further comprising:
outputting a first signal when the result of a comparison between characteristics of at least one of the digital images obtained by the imaging device and characteristics of the overlay image falls within a predetermined range for a degree of similarity; and/or
outputting a second signal when the result of the comparison between characteristics of at least one of the at least one digital image obtained by the imaging device and characteristics of the overlay image falls outside the predetermined range for a degree of similarity.

10. The method according to claim 1, further comprising:
automatically adapting the settings of the imaging device based on the result of a comparison between characteristics of at least one of the at least one digital images obtained by the imaging device and characteristics of the overlay image.

11. The method according to claim 1, further comprising:
obtaining identification information allocated to a patient wearing the transparent wound dressing and/or to the transparent wound dressing; and
saving the at least one digital image of the transparent wound dressing in association with the obtained identification information.

12. The method according to claim 1, further comprising:
obtaining wound information on a wound dressed with the transparent wound dressing; and
saving the at least one digital image of the transparent wound dressing in association with at least one piece of the obtained wound information.

13. A computer program comprising instructions, which, when executed by an apparatus, causes the apparatus to perform the method of claim 1.

14. A computer readable medium comprising program instructions stored thereon for performing the method of claim 1.

15. A method for assisting a user in reviewing digital images of a transparent wound dressing comprising at least the following steps:
obtaining at least two digital images of the transparent wound dressing captured according to the method of claim 1, wherein each digital image of the transparent wound dressing is associated with a respective chronological indicator;
providing a slider wherein the slider can adopt as many positions as digital images of the transparent wound dressing have been obtained, and wherein one position of the slider is assigned to one chronological indicator of the obtained at least two digital images of the transparent wound dressing; and
displaying the one digital image of the at least two digital images of the transparent wound dressing whose chronological indicator is assigned to the current position of the slider.

16. A computer program comprising instructions, which, when executed by an apparatus, causes the apparatus to perform the method of claim 15.

17. a computer readable medium comprising program instructions stored thereon for performing the method of claim 15.

18. A system for assisting a first user in capturing a digital image of a transparent wound dressing and for assisting a second user in reviewing digital images of a transparent wound dressing comprising at least the following:
a first mobile device having a computer readable medium, an imaging device, and a user interface, the computer readable medium including program instructions stored thereon for performing a method for assisting a user in capturing a digital image of a transparent wound dressing, preferably for monitoring the healing process of a wound dressed with and visible through the transparent wound dressing, the method including at least the following steps:
obtaining, by means of an imaging device, at least one digital image of the transparent wound dressing, and displaying, by means of a user interface, the obtained at least one digital image of the transparent wound dressing;
superimposing an overlay image on the at least one digital image of the transparent wound dressing displayed by the user interface; and
saving at least one digital image of the transparent wound dressing;
a second device having a computer readable medium according to claim 17 and a user interface; and
a server adapted for obtaining from the first mobile device at least one digital image (14, of a transparent wound dressing captured by the first mobile device, for storing the obtained at least one digital image of a transparent wound dressing captured by the first mobile device, and for transmitting the stored at least one digital image of a transparent wound dressing captured by the first mobile device to the second device.
